(19) United States
(12) Patent Application Publication
(10) Pub. No.: US 2022/0262464 A1
(43) Pub. Date: Aug. 18, 2022

(54) DEVICE AND METHOD FOR DETERMINING A PROPERTY OF A SUBSTANCE OR A MATERIAL

(71) Applicant: Robert Bosch GmbH, Stuttgart (DE)

(72) Inventors: Dominic Lingenfelser, Hambruecken (DE); Elisabeth Lotter, Pforzheim (DE)

(21) Appl. No.: 17/628,965

(22) PCT Filed: Aug. 14, 2020
(86) PCT No.: PCT/EP2020/072853

§ 371 Date: Jan. 21, 2022

(30) Foreign Application Priority Data
Aug. 19, 2019 (DE) 10 2019 212 332.5

(51) Int. Cl. G16C 20/30; G06N 5/02; G16C 20/70

(52) U.S. Cl. CPC G16C 20/30; G16C 20/70; G06N 5/022

(57) ABSTRACT

A device and method for determining a property of a substance or a material, in particular, the viscosity of an adhesive agent. A model is provided which includes a plurality of modules, which are configured to process data as a function of input variables. A module of the plurality of modules is configured to determine output variables, which characterize the properties of substances or materials. In one selection, at least one of the output variables, which is determined, being selectable. The selection is identified, and at least one of the input variables, whose data are processed by the model, being determined as a function of the selection. At least one of the modules is selected to process data for the at least one of the input variables.

### FIELD

The present invention relates to a device and a method for determining a property of a substance or a material.

### BACKGROUND INFORMATION

In order to ensure uniform product quality and also reduce costs for material, it is desirable to detect, early on, product differences resulting from unknown fluctuations, for example, between different batches, or due to deliberate manipulations or changes, such as from product counterfeiting.

### SUMMARY

In accordance with an example embodiment of the present invention, a method is provided for determining a property of a substance or a material, in particular, the viscosity of an adhesive agent. The method provides for a model to include a plurality of modules, which are configured to process data as a function of input variables; one module of the plurality of modules being configured to determine output variables, which characterize the properties of substances or materials; in one selection, at least one of the output variables, which is determined, being selectable; the selection being identified, and at least one of the input variables, whose data are processed by the model, being determined as a function of the selection; at least one of the modules being selected to process data for the at least one of the input variables. By this selection of the input variables as a function of the property to be determined, indirect process and material parameters, which are not accessible by conventional evaluation methodology, may be determined by machine learning algorithms with the aid of sensorial or analytical data. This allows product differences, manipulations or changes to be identified in a simple, precise, and reasonable manner.

In accordance with an example embodiment of the present invention, as a function of the selection, or as a function of the at least one of the input variables, at least one of the modules, which evaluates preprocessed data as a function of data regarding the at least one of the input variables, is preferably determined; the at least one of the output variables being determined as a function of the preprocessed data. In this manner, data preprocessing, which is adapted to the property to be determined or the respective input variables, is selected. This permits elimination of disturbance variables, device independence, or a dimensional or feature selection.

One module includes, for example, a plurality of predefined classification and/or regression models; at least one classification model and/or at least one regression model being selected as a function of the selection, in order to determine the at least one of the output variables. This modular model may be configured particularly easily.

In accordance with an example embodiment of the present invention, a partial least squares regression is preferably selected for determining an output variable, which characterizes a viscosity of an adhesive agent. This model may effectively process linear relationships in a multidimensional data space and has little tendency towards overfitting.

In accordance with an example embodiment of the present invention, at least one of the input variables preferably characterizes spectral data, thermoanalytic method data, or rheological method data. These are suitable for supplying information about a plurality of substances or materials. Due to this, the model may be used in a versatile manner.

In accordance with an example embodiment of the present invention, for the selection of an output variable, which characterizes a viscosity of an adhesive agent, an input variable, which characterizes spectral data in the mid-infrared range (FTIR), is preferably selected. This input variable contains a large amount of information regarding the molecular composition of an adhesive agent and is particularly suitable for the application of machine learning due to the high dimensionality of the data.

In accordance with an example embodiment of the present invention, for the selection of an output variable, which characterizes a viscosity of an adhesive agent, a module for preprocessing at least one input variable is preferably selected, which includes Sawitzky-Golay filtering for minimizing noise and an SNV transformation for computationally eliminating an offset from the data of this input variable. The resulting preprocessed data are particularly well-suited for determining the viscosity of an adhesive agent.

In accordance with an example embodiment of the present invention, for the selection of an output variable, which characterizes a viscosity of an adhesive agent, a module, which is configured to carry out removal of disturbance variables, using error removal by orthogonal subtraction (EROS), is preferably selected for preprocessing at least one input variable. In this manner, variances, which do not come from the output variable, are eliminated. This increases the robustness of the model.

In accordance with an example embodiment of the present invention, for the selection of an output variable, which characterizes a viscosity of an adhesive agent, a module for preprocessing at least one input variable is preferably selected, which is configured to carry out stepwise variable selection, in order to select the at least one input variable that correlates the most with the output value. This feature selection increases the predictive accuracy of the model.

In accordance with an example embodiment of the present invention, a device is provided for determining a parameter, in particular, of an adhesive agent, includes a plurality of processors, as well as at least one storage device for a model, which are configured to carry out the method.

Further advantageous specific embodiments of the present invention are derived from the following description and the figures.

### BRIEF DESCRIPTION OF THE DRAWINGS

FIG. 1 shows a device for determining a parameter, in accordance with an example embodiment of the present invention.

FIG. 2 shows a method for determining a parameter, in accordance with an example embodiment of the present invention.

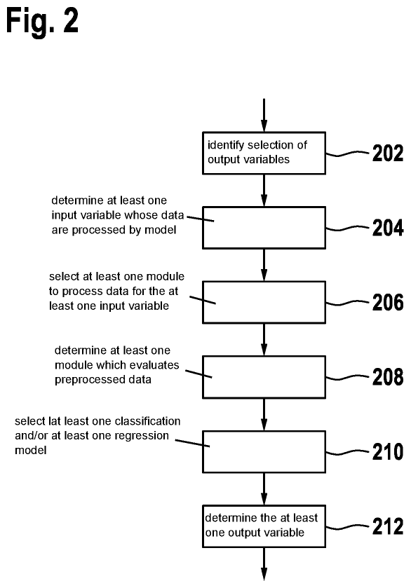

### DETAILED DESCRIPTION OF EXAMPLE EMBODIMENTS

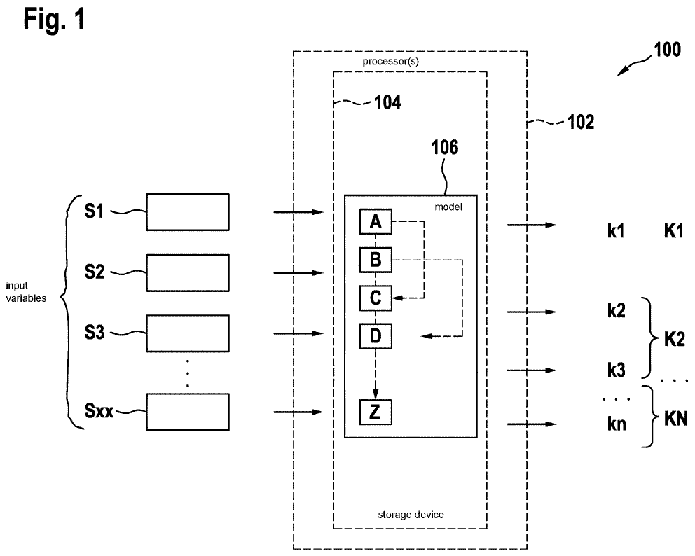

A device 100 regarding a property of a substance or a material, in particular, the viscosity of an adhesive agent, is represented schematically in FIG. 1. The determination of a plurality of properties k1, k2, . . . , kn from different categories K1, K2, . . . , KN is provided in the example. Device 100 includes a plurality of processors 102 and a storage device 104 for a model 106. Device 100 is configured to carry out the method described in the following. A powerful computer may be provided, which is configured to determine parameters of model 106.

By combining sensorial/analytic data from input variables, which are denoted by S1, . . . , Sxx in FIG. 1, indirect process and material parameters, which are characterized in FIG. 1 by the properties k1, k2, . . . , kn from different categories K1, K2, . . . , KN, are determined by machine learning algorithms, which are represented by model 106. These are not accessible by conventional evaluation methodology.

The data of input variables S1, . . . , Sxx may include analytical methods, such as spectral data, which are denoted by S1 in FIG. 1, thermoanalytic methods, which are denoted by S2 in FIG. 1, and rheological data, which are denoted by S3 in FIG. 1.

Model 106 includes modules, which are indicated by A through Z in FIG. 1. Model 106 is made up of different machine learning algorithms. In module A, these include preprocessing of data, which are adapted to the specific data of input variables S1, . . . , Sxx. In module B, these include elimination of disturbance variables for device independence. In module C, these include dimensional or feature selection. In module D, these include classification and/or regression algorithms.

Properties k1, k2, . . . , kn are categorized in superordinate categories K1, K2, . . . , KN. In the example, category K1 includes material-specific properties. In the example, category K2 includes parameters, which change over the product life cycle. In the example, category 3 includes mechanical parameters. In the example, category 4 includes categorized data, which are divided into subcategories. For example, subcategories include specific parameters, which may be acquired from, or described more precisely by, either one of the input variables S1, . . . , Sxx or a combination of a plurality of input variables S1, . . . , Sxx.

The following points come into consideration for use in adhesive agent technology:

Input variables S1, . . . , Sxx:

S1: spectral data (300 nm . . . 3 mm), e.g., UV vis., near-infrared (NIR), mid-infrared (FTIR), far-infrared (terahertz), Raman spectroscopy, chemiluminescence.

S2: thermoanalytic methods, such as thermogravimetry, differential thermoanalysis (DSC),

S3: rheological methods.

Models:

Module A: preprocessing, e.g., detrending, derivation, mean centering, Savitzky-Golay filtering, Fourier transformation, standard normal variate (SNV).

Module B: elimination of disturbance variables for device independence, e.g., error removal by orthogonal subtraction (EROS), external parameter orthogonalization (EPO), wavelet transformation, Fourier transformation.

Module C: dimensional reduction or feature selection, e.g., principal component analysis (PCA) for dimensional reduction, stepwise variable selection (SVS), Procrustes variable selection.

Module D: classification and/or regression algorithms, e.g., partial least squares regression/classification (PLS-Reg, PLS-DA), linear discriminant analysis (LDA), ridge regression, multiple linear regression (MLR), logistic regression, decision and regression tree, random forest, support vector machine (SVM), artificial neural networks (ANN).

Output Variables:

Category 1: chemical composition, e.g., type of polymer, additives, ratio of the components, catalysts.

Category 2: material properties, e.g., water content in the material (xH2O), morphology, flowability, degree of cross-linking, viscosity of the material, reactivity, monomer content, acid value.

Category 3: mechanical quantities, such as imperviousness of the adhesive, combined tension and shear resistance.

Category 4: categorized, e.g., differences (setpoint/actual), adhesion (effective/poor).

In the following, one variant is described as an example for the regression of the viscosity of adhesive agents. The combination of input variables, modules and output variables described in the following are suited for this.

A method for determining a property of a substance or a material is described below, using the example of the viscosity of an adhesive agent.

Model 106 includes a plurality of modules A, . . . , Z, which are configured to process data as a function of input variables S1, . . . , Sxx.

Module D is configured to determine output variables k1, . . . , kn, which characterize properties of substances or materials.

In one selection, at least one of output variables k1, . . . , kn, which is determined by module D, is selectable.

In the method, in a step 202, the selection is identified, and the following configuration of model 106 is to be taken up as a function of the selection:

In a step 204, at least one of input variables S1, . . . , Sxx is determined, whose data are processed by model 106. The at least one of input variables S1, . . . , Sxx is selected from input variables, which characterize spectral data, thermoanalytic method data, or rheological method data. For the selection of an output variable k1, . . . , kn, which characterizes a viscosity of an adhesive agent, an input variable S1, which characterizes spectral data in the mid-infrared range (FTIR), is selected.

In a step 206, at least one of modules A, . . . , Z is selected to process data for the at least one of input variables S1, . . . , Sxx. For the selection of an output variable k1, . . . , kn, which characterizes a viscosity of an adhesive, a module A for preprocessing at least one input variable S1 is selected, which includes Sawitzky-Golay filtering for minimizing noise and an SNV transformation for computationally eliminating an offset from the data of this input variable S1. For the selection of an output variable k1, . . . , kn, which characterizes the viscosity of an adhesive agent, a module B, which is configured to carry out removal of disturbance variables, using error removal by orthogonal subtraction (EROS), is selected for preprocessing at least one input variable S1. For the selection of output variable k1, . . . , kn, which characterizes the viscosity of an adhesive agent, a module C for preprocessing at least one input variable S1 is selected, which is configured to carry out stepwise variable selection, in order to select the at least one input variable S1 that correlates the most with the output variable.

After the preprocessing of input variable S1, the preprocessed data are available to module D for determining the at least one output variable k1, . . . , kn, which characterizes the viscosity of an adhesive agent.

In a step 208, as a function of the selection, or as a function of the at least one of the input variables S1, . . . , Sxx, at least one of modules A, . . . , Z, which evaluates preprocessed data as a function of data regarding the at least one of input variables S1, . . . , Sxx, is determined.

In a step 210, at least one classification and/or at least one regression model for determining the at least one of output variables k1, . . . , kn is selected for module D from a plurality of predefined classification and/or regression models as a function of the selection. A partial least squares regression is selected for determining an output variable k1, . . . , kn, which characterizes a viscosity of an adhesive agent.

In a step 212, the at least one of output variables k1, . . . , kn is determined as a function of the preprocessed data.

This model 106 may be trained in a training method as a function of labeled training data.

1-12. (canceled)

13. A method for determining a property of a substance or a material, wherein a model is provided which includes a plurality of modules which are configured to process data as a function of input variables, a module of the plurality of modules being configured to determine output variables which characterize properties of substances or materials, at least one of the output variables to be determined being selectable, the method comprising the following steps:
identifying a selection of the at least one of the output variables;
determining at least one of the input variables whose data are processed by the model, as a function of the selection; and
selecting at least one of the modules to process data for the determined at least one of the input variables.

14. The method as recited in claim 13, wherein the property of the substance or the material is a viscosity of an adhesive agent.

15. The method as recited in claim 13, wherein as a function of the selection, or as a function of the determined at least one of the input variables, at least one of the modules, which evaluates preprocessed data as a function of data regarding the determined at least one of the input variables, is determined, and the at least one output variable being determined as a function of the preprocessed data.

16. The method as recited in claim 13, wherein a module of the plurality of modules includes a plurality of predefined classification and/or regression models, and at least one classification model and/or at least one regression model for determining the at least one of the output variables is selected as a function of the selection.

17. The method as recited in claim 16, wherein a partial least squares regression is selected for determining an output variable of the at least one output variable, which characterizes a viscosity of an adhesive agent.

18. The method as recited in claim 13, wherein at least one of the input variables characterizes spectral data, or thermoanalytic method data, or rheological method data.

19. The method as recited in claim 13, wherein for the selection of an output variable of the at least one of the output variable, an input variable, which characterizes spectral data in the mid-infrared range, is selected, the output variables characterizing a viscosity of an adhesive agent.

20. The method as recited in claim 13, wherein for the selection of an output variable of the at least one output variable, a module for preprocessing at least one input variable is selected which includes Sawitzky-Golay filtering for minimizing noise and an SNV transformation for computationally eliminating an offset from the data of the at least one input variable, wherein the output variable characterizes a viscosity of an adhesive agent.

21. The method as recited in claim 13, wherein for the selection of an output variable of the at least one output variable, a module which is configured to carry out removal of disturbance variables, using error removal by orthogonal subtraction, is selected for preprocessing at least one of the at least one input variable, wherein the output variable characterizes a viscosity of an adhesive agent.

22. The method as recited in claim 13, wherein for the selection of an output variable of the at least one of the output variables, a module for preprocessing at least one input variable is selected, which is configured to carry out stepwise variable selection, in order to select the at least one input variable that correlates the most with output variable, the output variable characterizing a viscosity of an adhesive agent

23. A device for determining a parameter of an adhesive agent, the device comprising:
a plurality of processors; and
at least one storage device for a model, the model including a plurality of modules which are configured to process data as a function of input variables, a module of the plurality of modules being configured to determine output variables which characterize properties of substances or materials, at least one of the output variables to be determined being selectable;
wherein the device is configured to:
identify a selection of the at least one of the output variables,
determine at least one of the input variables whose data are processed by the model, as a function of the selection, and
select at least one of the modules to process data for the determined at least one of the input variables.

24. A non-transitory machine-readable storage medium on which is stored a computer program including computer-readable instructions for determining a property of a substance or a material, wherein a model is provided which includes a plurality of modules which are configured to process data as a function of input variables, a module of the plurality of modules being configured to determine output variables which characterize properties of substances or materials, at least one of the output variables to be determined being selectable, the instructions, when executed by a computer, causing the computer to perform the following steps:
identifying a selection of the at least one of the output variables;
determining at least one of the input variables whose data are processed by the model, as a function of the selection; and
selecting at least one of the modules to process data for the determined at least one of the input variables.
